(19) United States
(12) Patent Application Publication
(10) Pub. No.: US 2008/0176934 A1
(43) Pub. Date: Jul. 24, 2008

(54) Use Of Diosmetin Compounds In The Treatment And Prevention Of Thrombotic Pathologies

(75) Inventors: Tony Verbeuren, Vernouillet (FR); Alain Rupin, Savonnieres (FR); Patricia Sansilvestri-Morel, Antony (FR); Marie-Odile Vallez, Montreuil (FR); Marie-Francoise Boussard, Mareil-Sur-Mauldre (FR); Michel Wierzbicki, L'Etang-La-Ville (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: Les Laboratoires Servier, Courbevoie Cedex (FR)

(21) Appl. No.: 11/885,372

(22) PCT Filed: Feb. 28, 2006
(86) PCT No.: PCT/FR06/00441

§ 371 Date: Aug. 30, 2007

(30) Foreign Application Priority Data
Mar. 1, 2005 (FR) 0502044

(51) Int. Cl. A61K 31/352; A61P 9/00

(52) U.S. Cl. 514/456

(57) ABSTRACT

Use of diosmetin compounds in obtaining pharmaceutical compositions intended for the prevention and/or treatment of thrombotic pathologies and pathologies with a risk of thrombosis.

The present invention relates to use of diosmetin compounds in obtaining pharmaceutical compositions intended for the prevention and/or treatment of thrombotic pathologies and of pathologies with a risk of thrombosis.

The invention relates in particular to use of the diosmetin compound 6,8-diallyl-5,7-dihydroxy-2-(2-allyl-3-hydroxy-4-methoxyphenyl)-4H-1-benzo-pyran-4-one in obtaining pharmaceutical compositions intended for the prevention and/or treatment of thrombotic pathologies.

The physiological process of haemostasis is triggered by an adverse change in the endothelial cells of the blood vessels, either for incidental reasons or for more complex pathological reasons. Its purpose is to block and stem the escape of blood by means of two steps that are different from one other but related to and dependent on one another: primary haemostasis and plasma coagulation.

Primary haemostasis is the emergency mechanism involving circulating blood platelets which adhere to the endothelium to form the white thrombus or platelet plug.

Secondary thereto, the platelet thrombus is strengthened by the formation of a network of fibrin which wraps the aggregated platelets within its meshes. The insoluble fibrin is generated starting from a soluble plasma protein, fibrinogen, under the action of thrombin, the final product of the enzymatic activation cascade of the coagulation system.

Finally, the fibrin/platelet thrombus is resorbed by the phenomenon of fibrinolysis. In fact, because of its capacity to reduce the amount of fibrin in the vascular circulation, fibrinolysis allows the body to combat the occurrence of thromboses as well as to break down a thrombus that has initially stopped a haemorrhage.

Fibrinolysis principally involves plasmin, a proteolytic enzyme which breaks down the long chains of polymerised fibrin into D-dimers. This enzyme is present in plasma in the form of a zymogen, plasminogen, which is activated into plasmin by various serine proteases present on the surface of the fibrin/platelet clot. These serine proteases are, in particular, plasminogen activators of the tissue type (t-PA), which are essentially released by the activated endothelial cells, and of the urokinase type (u-PA), whose release in the form of pro-urokinases is much more ubiquitous.

Fibrinolysis is itself negatively regulated by a three-component system comprising plasmin inhibitors (α₂-antiplasmin and α₂-macroglobulin), plasminogen activation inhibitors (PAI-1 or type 1 Plasminogen Activator Inhibitor; PAI-2) and thrombin-activable fibrinolysis inhibitor (TAFI).

The principal plasminogen activator inhibitor is PAI-1. The PAI-1 protein, which belongs to the super-family of serpins (serine protease inhibitors), is a glycoprotein of 379 amino acids (47 kDa) lacking in cysteine but very rich in methionine residues. The PAI-1 protein, which is produced by numerous types of cells such as endothelial cells, monocytes, hepatocytes, fibroblasts, adipocytes and megakaryocytes, is present in plasma and platelets. The active site of PAI-1, located at the C-terminal end of the protein at the position (Arg³⁴⁶-Met³⁴⁷), behaves like a pseudo-substrate for tissue-type plasminogen activators. The principal target proteins of PAI-1 are therefore t-PA and u-PA.

Taking into account the mechanisms regulating haemostasis and fibrinolysis on the scale of the body as a whole, a positive correlation has been shown between a raised level of PAI-1 and an increased risk of venous and arterial thromboses. This positive correlation is reported, in particular, in the case of pathologies whose origin is venous or arterial thrombosis such as myocardial infarction (Hamsten et al. Plasminogen activator inhibitor in plasma: risk factor for recurrent myocardial infarction, 1987, Lancet 2, 3-9), angina (Wieczorek et al. Tissue-type plasminogen activator and tissue plasminogen activator inhibitor activities as predictor of adverse events in unstable angina, 1994, Am J Cardiol, 74, 424-429), intermittent claudication, cerebral vascular accidents, deep vein thrombosis (Schulman et al. The significance of hypofibrinolysis for the risk of recurrence of venous thromboembolism, 1996, Thromb Haemost, 75, 307-611), pulmonary embolism and also pathologies in which the risk of thromboses is increased such as hypertension, hypercholesterolaemia, diabetes, obesity, genetic abnormalities of blood coagulation or acquired abnormalities of blood coagulation.

The plasma level of PAI-1 is, in fact, raised in 30% of patients affected by venous thromboembolism (Tabernero et al. Incidence of increased plasminogen activator inhibitor in patients with deep venous thrombosis and/or pulmonary embolism, 1989, Thromb Res, 56, 565-570). The consequence of that is general dysfunction of the fibrinolytic system and especially a drop in t-PA. The same observations have been made in patients suffering from pulmonary hypertension subsequent to an embolism (Huber et al. Fibrinogen, t-PA and PAI-1 plasma levels in patients with primary pulmonary hypertension, 1994, Am J Crit. Care Med, 150, 929-933). In that particular case, the increase in the level of PAI-1 is due to endothelial cells located in the thrombosis zone which exhibit an increase in the release of PAI-1 that is associated with induction either by thrombin or by a mediator released by the platelets.

Given that a raised level of PAI-1 is deleterious in the context of cardiovascular syndromes it has been considered that restoring normal fibrinolytic activity would prevent the occurrence of thrombotic events in patients at risk.

Control of the fibrinolysis process accordingly constitutes a central problem in medical cardiovascular pathologies.

Anticoagulants and antiplatelets constitute customary treatments in a wide range of cardiovascular pathologies. Their value in the context of preventing cardiovascular accidents and also for cardiovascular emergencies has been demonstrated in epidemiological studies. However, the risk/benefit ratio must always be weighed up because accidents associated with anticoagulants and antiplatelets are dominated by haemorrhages which can call into question the functional or vital prognosis. Indeed, the principal complication of anticoagulant and antiplatelet treatments is bleeding.

Anticoagulants administered by the parenteral route are, in particular, non-fractionated heparin (NFH), fractionated heparins (LMWH) and acenocoumarol (Sintrom®). Epidemiological studies have shown that the therapeutic class of fractionated and non-fractionated heparins in the context of the treatment of a thromboembolic episode causes major bleeding in 0 to 7% of cases with a mortality ranging from 0 to 2%. The anticoagulant Sintrom®, developed in prophylactic and curative treatment of thromboembolic symptoms, also gives rise to major life-threatening haemorrhagic events in up to 2.2% of cases. Taking into account the critical increase in haemorrhagic risks in the combination of Sintrom® with an antiplatelet such as aspirin, the risk/benefit ratio of that combination must not be underestimated when making therapeutic choices.

More recent treatments having a fibrinolytic or thrombolytic action have been developed; they accelerate the dissolution of intravascular clots by promoting the conversion of the inactive plasminogen into active plasmin (Marder V J., Thrombolytic therapy: foundations and clinical results in <<Haemostasis and Thrombosis>>, Fourth Edition 2001, Editors Colman R W et al.). These fibrinolytics or thrombolytics are administered exclusively by the parenteral route, either by the general intravenous route in perfusion or as a bolus dose, or by a local, e.g. intracoronary or intra-arterial, route. These pharmaceutical compositions must, moreover, be administered as early as possible after formation of the thrombus in order to try to dissolve it and thereby remove the obstruction in the vessel. These new thrombolytics accordingly consist of analogues of tissue plasminogen activator or t-PA such as, for example: alteplase, which is obtained by genetic engineering and is identical to t-PA; reteplase, which is a simplified analogue of human t-PA; or tenecteplase, which is a recombinant protein similar to endogenous t-PA and which has greater affinity for the fibrin of the thrombus. These fibrinolytics were quick to show their limitations in respect of their emergency hospital use and their problems of administration, for example introduction of a catheter into the thrombosed artery.

Other potential therapies have been envisaged in order to modify the level of PAI-1, the increase in which is correlated with thromboembolic accidents. In particular, monoclonal antibodies have been developed as inhibitors of the activity of PAI-1. MAI 12 is a murine antibody specific to human PAI-1, which does not interfere either with PAI-2 or with t-PA or α2-antiplasmin. By blocking the interaction of PAI-1 with t-PA, this monoclonal antibody increases fibrinolysis in vitro and in vivo (Levi et al. Inhibition of plasminogen activator inhibitor-1 activity results in promotion of endogenous thrombolysis and inhibition of thrombus extension in models of experimental thrombosis, 1992, Circulation, 85, 305-312). In the case of the monoclonal antibody CLB-2C, this antibody binds between positions 128 and 145 in the amino acid sequence of vitronectin and accordingly prevents the protein PAI-1 from binding thereto, thereby accelerating its inactivation. Despite the effectiveness of these monoclonal antibodies in in vitro and in vivo models, these specific inhibitors of PAI-1 cannot be exploited from a medical point of view because of the difficulties associated with the lack of humanisation of the antibodies, the risks of immunogenicity and the costs of development.

The present invention is accordingly aimed at proposing an alternative strategy to the already available mechanisms for modifying fibrinolysis, for preventive and curative purposes with respect to thrombotic pathologies, with a view to overcoming at least in part the disadvantages of known treatments for thromboembolic accidents. This alternative strategy is based on inhibition of the expression of the PAI-1 gene.

To that effect, the invention proposes the use of diosmetin compounds in obtaining pharmaceutical compositions intended for the prevention and/or treatment of thrombotic pathologies. The diosmetin compounds according to the invention are compounds of the general formula (I):

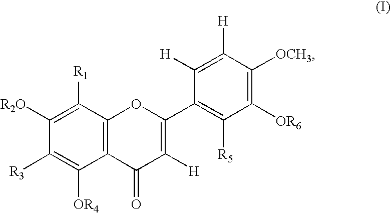

wherein
R₁ represents a hydrogen atom or a propyl or allyl radical;
R₂ represents a hydrogen atom or a propyl, allyl, 2,3-dihydroxypropyl, (2,2-dimethyl-1,3-dioxol-4-yl)methyl or 3-acetyloxy-2-hydroxypropyl radical;
R₃ represents a hydrogen atom or a propyl or allyl radical;
R₄ represents a hydrogen atom or a methyl, propyl, allyl, 2,3-dihydroxypropyl or (2,2-dimethyl-1,3-dioxol-4-yl)methyl radical or a radical of formula —COR′₄ wherein R′₄ represents a linear or branched (C₁-C₅)alkyl radical or a phenyl radical;
R₅ represents a hydrogen atom or a propyl or allyl radical, and
R₆ represents a hydrogen atom or a methyl, propyl, allyl, 2,3-dihydroxypropyl or (2,2-dimethyl-1,3-dioxol-4-yl)methyl radical, a radical of formula —COR′₆ (wherein R′₆ represents a linear or branched (C₁-C₅)alkyl radical or a phenyl radical) or a radical of formula (I′)

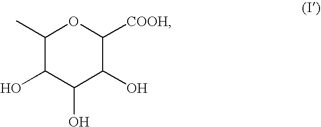

provided that:
at least one of the groups R₁, R₂, R₃, R₄, R₅ and R₆ is other than a hydrogen atom and,
if R₁, R₂ and R₃ each simultaneously represent a hydrogen atom, then R₄ also represents a hydrogen atom,
   their diastereoisomers and enantiomers, and also
   addition salts thereof with a pharmaceutically acceptable acid or base.

Preparation of the Compounds of the General Formula (I) is Described in the patent specification EP 0 709 383.

More particularly, the diosmetin compounds used according to the invention are the following compounds, described in the patent specification EP 0 709 383, of formulae (II) to (XVII):
(II) 7-allyloxy-5-hydroxy-2-(3-hydroxy-4-methoxyphenyl)-4H-1-benzopyran-4-one

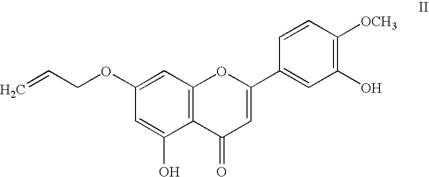

(III) 5,7-dihydroxy-2-[3-(2,3-dihydroxypropyloxy)-4-methoxyphenyl]-4H-1-benzopyran-4-one

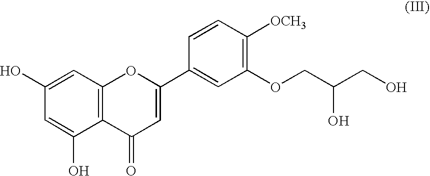

(IV) (R,S)-5-hydroxy-2-(3-hydroxy-4-methoxyphenyl)-7-(2,3-dihydroxypropyloxy)-4H-1-benzopyran-4-one

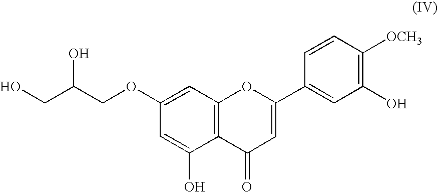

(V) 5,7-di-(2,3-dihydroxypropyloxy)-2-(3-hydroxy-4-methoxyphenyl)-4H-1-benzopyran-4-one

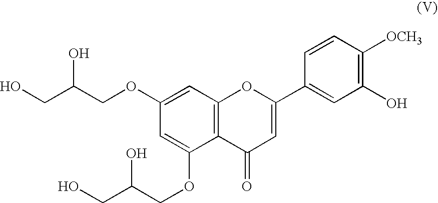

(VI) 5-hydroxy-2-(4-methoxy-3-pivaloyloxyphenyl)-7-(2,3-dihydroxypropyloxy)-4H-1-benzopyran-4-one

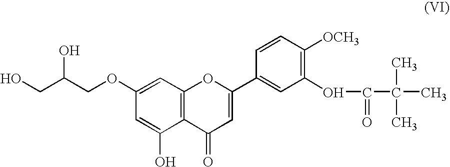

(VII) 5-allyloxy-2-(3-allyloxy-4-methoxyphenyl)-7-(2,3-dihydroxypropyloxy)-4H-1-benzopyran-4-one

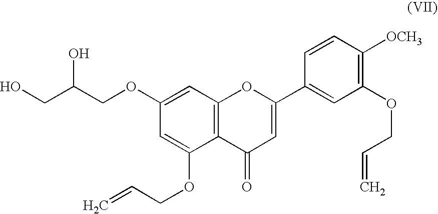

(VIII) 6-allyl-5-hydroxy-2-(2-allyl-3-hydroxy-5-methoxyphenyl)-7-(2,3-dihydroxypropyloxy)-4H-1-benzopyran-4-one

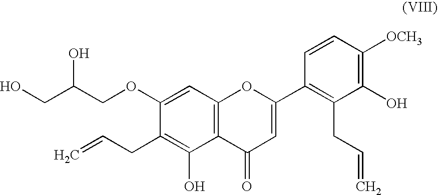

(IX) 5-hydroxy-2-(3-hydroxy-4-methoxy-2-propylphenyl)-6-propyl-7-(2,3-dihydroxypropyloxy)-4H-1-benzopyran-4-one

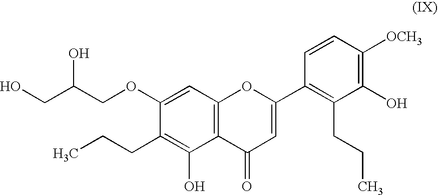

(X) (R,S)-5-hydroxy-2-(3-hydroxy-4-methoxy-2-propylphenyl)-7-(2,3-dihydroxypropyloxy)-4H-1-benzopyran-4-one

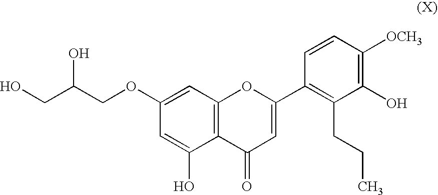

(XI) (R)-5-hydroxy-2-(3-hydroxy-4-methoxy-2-propylphenyl)-7-(2,3-dihydroxypropyloxy)-4H-1-benzopyran-4-one
(XII) (S)-5-hydroxy-2-(3-hydroxy-4-methoxy-2-propylphenyl)-7-(2,3-dihydroxypropyloxy)-4H-1-benzopyran-4-one
(XIII) (R,S)-5-hydroxy-2-(3-hydroxy-4-methoxy-2-propylphenyl)-7-(3-acetyloxy-2-hydroxypropyloxy)-4H-1-benzopyran-4-one

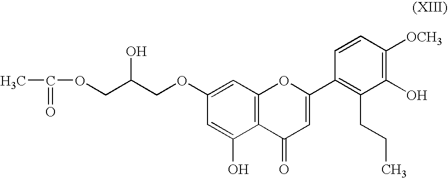

(XIV) 5-hydroxy-2-[4-methoxy-2-propyl-3-(6-carboxy-3,4,5-trihydroxytetrahydropyran-2-yl-oxy)-phenyl]-7-(2,3-dihydroxypropyloxy)-4H-1-benzopyran-4-one

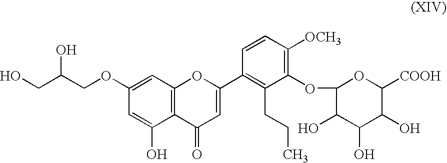

(XV) 5-hydroxy-2-[4-methoxy-3-(2,3-dihydroxypropyloxy)-phenyl]-7-(2,3-dihydroxypropyloxy)-4H-1-benzopyran-4-one

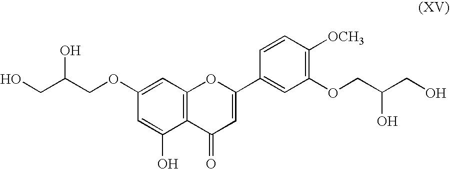

(XVI) 5,7-dihydroxy-2-(3-hydroxy-4-methoxy-2-propylphenyl)-6,8-dipropyl-4H-1-benzopyran-4-one

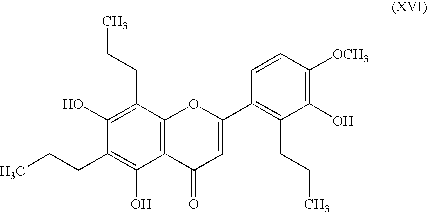

More advantageously, the preferred compound of formula (I) according to the invention is 6,8-diallyl-5,7-dihydroxy-2-(2-allyl-3-hydroxy-4-methoxyphenyl)-4H-1-benzopyran-4-one of formula (XVII):

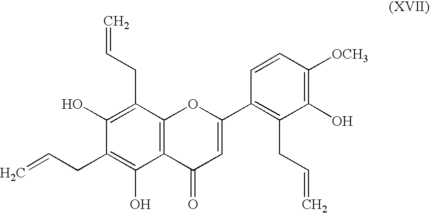

The present invention accordingly relates to the use of diosmetin compounds of formula (I):

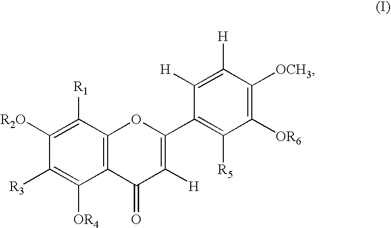

wherein:
R₁ represents a hydrogen atom or a propyl or allyl radical;
R₂ represents a hydrogen atom or a propyl, allyl, 2,3-dihydroxypropyl, (2,2-dimethyl-1,3-dioxol-4-yl)methyl or 3-acetyloxy-2-hydroxypropyl radical;
R₃ represents a hydrogen atom or a propyl or allyl radical;
R₄ represents a hydrogen atom or a methyl, propyl, allyl, 2,3-dihydroxypropyl or (2,2-dimethyl-1,3-dioxol-4-yl)methyl radical or a radical of formula —COR′₄ wherein R′₄ represents a linear or branched (C₁-C₅)alkyl radical or a phenyl radical;
R₅ represents a hydrogen atom or a propyl or allyl radical, and
R₆ represents a hydrogen atom or a methyl, propyl, allyl, 2,3-dihydroxypropyl or (2,2-dimethyl-1,3-dioxol-4-yl)methyl radical, a radical of formula —COR′₆ (wherein R′₆ represents a linear or branched (C₁-C₅)alkyl radical or a phenyl radical) or a radical of formula (I′)

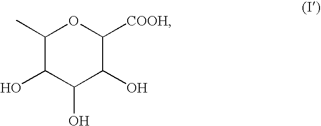

provided that:
at least one of the groups R₁, R₂, R₃, R₄, R₅ and R₆ is other than a hydrogen atom and,
if R₁, R₂ and R₃ each simultaneously represent a hydrogen atom, then R₄ also represents a hydrogen atom,
   their diastereoisomers and enantiomers, and also
   addition salts thereof with a pharmaceutically acceptable acid or base,
   in obtaining pharmaceutical compositions intended for the prevention and/or treatment of thrombotic pathologies.

In accordance with the usual meaning in the framework of the invention, the expression “thrombotic pathologies” is defined as any pathology brought about by the formation of a thrombosis in the cardiovascular system (veins, arteries, heart, micro-circulation).

Local obstruction of the vessel by a clot or obstruction at a remote location resulting from embolisation of the clot are responsible for the clinical signs of thrombotic pathologies. Deep vein thromboses appear essentially in the legs; if they embolise to the lungs they will cause a pulmonary embolism. Arterial thromboses occur most frequently in association with a vascular pathology, the most common being atherosclerosis. They are responsible for tissue ischaemia by stopping blood flow in the artery, for example myocardial infarction in the case of a coronary thrombosis or of a thrombosis further down in the micro-circulation after embolisation, or in the context of, for example, a cerebral vascular accident (CVA) after embolisation of an intracardiac or carotid thrombus. Finally, micro-circulatory thrombosis is generally a complication of disseminated intravascular coagulation in which micro-thrombi produce ischaemic necrosis.

The term “preventive” according to the invention corresponds to a preventively aimed treatment having the objective of reducing the risk of developing a thrombosis in predisposing contexts such as atherosclerotic diseases, diabetes or metabolic syndrome. In addition, the term “preventive” can be understood as secondary prevention which is intended to diminish prevalence by reducing the development and duration of the disease. Secondary prevention is essential following cardiovascular accidents and cerebral vascular accidents in order to reduce the risk of recurrence.

“Treatment” is understood as curatively aimed treatment prescribed for the purposes of treating a thrombosis which may or may not be complicated by an embolism.

The invention preferably relates to the use of 6,8-diallyl-5,7-dihydroxy-2-(2-allyl-3-hydroxy-4-methoxyphenyl)-4H-1-benzopyran-4-one of formula (XVII):

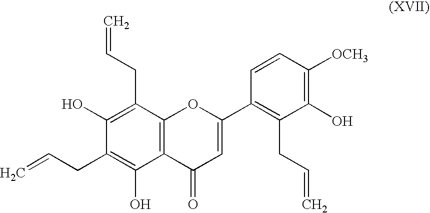

in obtaining pharmaceutical compositions intended for the prevention and/or treatment of thrombotic pathologies.

The present invention relates, moreover, to the use of diosmetin compounds in obtaining pharmaceutical compositions intended for the prevention and/or treatment of pathologies with a risk of thrombosis.

“Pathologies with a risk of thrombosis” are understood to be any pathology in the course of which thrombotic events occur. Pathologies which aggravate atherosclerosis such as hypercholesterolaemia, diabetes, hypertension, obesity, smoking and also atrial fibrillation are the principal pathologies with a risk of arterial thrombosis. Genetic or acquired abnormalities of coagulation proteins and orthopaedic surgery are the principal pathologies with a risk of venous thrombosis, but there may also be mentioned obesity, certain hormone treatments, certain cancers and their treatments, pregnancy and prolonged immobilisation causes. Finally, septic shock is the principal pathology with a risk of micro-circulatory thromboses.

For example, a raised level of triglycerides such as in hypercholesterolaemia is a particularly aggravating marker of cardiovascular risk in general and of thrombosis in particular. There is a correlation between a raised level of VLDL and of PAI-1 released by hepatocytes and endothelial cells (Allison et al. Effects of native triglyceride-enriched and oxidatively modified LDL on plasminogen activator inhibitor-1 expression in human endothelial cells, 1999, Arterioscler Thromb Vasc Biol, 19, 1354-1360) (Mussoni et al. Hypertriglyceridemia and regulation of fibrinolytic activity, 1992, Arterioscler Thromb, 12, 19-27).

The invention relates also to use of diosmetin compounds as an active ingredient in combination with one or more pharmaceutically acceptable excipients in obtaining pharmaceutical compositions intended for the prevention and/or treatment of thrombotic pathologies or pathologies with a risk of thrombosis.

The invention preferably relates to a pharmaceutical composition comprising a diosmetin compound according to the invention in combination with one or more pharmaceutically acceptable excipients, which composition is intended for the prevention and/or treatment of thrombotic pathologies or pathologies with a risk of thrombosis.

An “active ingredient” is understood to be any substance responsible for the pharmacodynamic or therapeutic properties of the pharmaceutical composition. In the context of the invention, “excipients” are understood to be any substance into which the active ingredient of a medicament is incorporated in order to facilitate the preparation and administration thereof and also to modify its consistency, form and volume.

The pharmaceutical compositions intended for the prevention and/or treatment of thrombotic pathologies or pathologies with a risk of thrombosis are moreover in a form that is suitable for oral, parenteral, nasal, per- or trans-cutaneous, rectal, perlingual, ocular or respiratory administration, especially tablets or dragées, sublingual tablets, sachets, paquets, capsules, glossettes, lozenges, suppositories, creams, ointments, dermal gels and drinkable or injectable ampoules.

The diosmetin compounds according to the invention are preferably used in obtaining pharmaceutical compositions for oral administration intended for the prevention and/or treatment of thrombotic pathologies or pathologies with a risk of thrombosis. Administration, by the oral route, of pharmaceutical compositions intended for thrombotic pathologies or pathologies with a risk of thrombosis is especially suitable for treatments having a preventive aim by virtue of the ease of patient administration.

Finally, the useful dosage varies according to the sex, age and weight of the patient, the administration route, the nature of the therapeutic indication and any associated treatments and ranges from 0.1 mg to 1 g per 24 hours in one or more administrations.

The present invention is illustrated by the following examples without being limited thereby.

### EXAMPLE 1

### In Vitro Inhibition of the Expression of PAI-1

1. Cell Culture

The study is carried out on a line of human hepatocytes HepG2 (ATCC, no. HB-8065). The cells are cultured in a medium of MEM (minimal essential medium) with glutamax-1 (Gibco™) containing 10% FCS (foetal calf serum), 1% non-essential amino acids and 1% sodium pyruvate and supplemented with penicillin and streptomycin.

2) Cloning of the PAI-1 Promoter

### a) Amplification of the Promoter by PCR

A fragment of 2.5 kb, corresponding to the PAI-1 promoter, extending from the nucleotides at positions −2442 to +136, was amplified by PCR and sub-cloned (Lopez et al., 2000, Atherosclerosis, 152, 359-366). In a final reaction volume of 50 μl, 2.5 units of Platinum® TaqDNA Polymerase High Fidelity (Invitrogen) are placed in the presence of 300 ng of human genomic DNA (Clontech), 300 μM of dNTP (deoxynucleotide triphosphate) (Clontech), 300 nM of primers in a medium containing the buffer specific to the enzyme, supplemented by 1.5 mM of MgCl₂ and different concentrations of PCRx Enhancer System (Invitrogen), which facilitates amplification of the especially difficult sequences (0 to 4×). The set of primers used (Chen et al. Differential mechanisms of PAI-1 gene activation by transforming growth factor beta and tumor necrosis factor alpha in endothelial cells, 2001, Thrmb Haemost, 86, 1563-72) is as follows:

| | |
|---|---|
| 5′-TTACGCGTGGGTTTGGGGCTGGACTTG-3′ | (SEQ ID NO.1) |
| and | |
| 5′-GGAGATCTCAGAGGTGCCTTGCGATTGG-3′. | (SEQ ID NO.2) |

The PCR program, on a Perkin Elmer 2400 apparatus, comprises initiation at elevated temperature at 94° C. for 2 min and then amplification over 35 cycles. The PCR product is then precipitated for 1 h at −80° C. in the presence of 7.5M ammonium acetate and ethanol. After centrifuging at 14000 rpm at 4° C., the sediment is resuspended in 70% ethanol and again centrifuged at 14000 rpm at 4° C. The sediment obtained is then dried and then taken up in water.

### b) Digestion of the Promoter Sequence

The amplified sequence is then digested in two steps by the restriction enzymes Bgl II and Mlu I. The amplified sequence is digested for 1 h 30 min at 37° C. in the presence of 1 unit/μl of enzyme and 100 μg/ml of BSA (Bovine Serum Albumin). After each digestion, the product obtained is systematically purified on Micro Bio-Spin® Chromatography columns (Bio-Rad) in order to remove the salts of the buffer.

### c) Construction of the PGL3/PAI-1 Plasmid

The pGL3-Basic plasmid (Promega), containing the luciferase gene of the firefly, is digested by the restriction enzymes Bgl II and Mlu I according to the same protocol as for the insert and is then purified on 1% low melting agarose gel.

Ligation of the pGL3-Basic plasmid vector and the insert corresponding to the PAI-1 promoter was carried out by means of T4 DNA Ligase (LigaFast™ Rapid DNA Ligation System from Promega). Conventionally, during ligation, there is used an excess of insert which is equivalent to three times the amount of vector. Furthermore, because this insert is half as long as the vector (2.5 kb as opposed to 4.8 kb), maintaining stoichiometric equilibrium requires twice the mass of vector. 37.5 ng of insert and 25 ng of pGL3-Basic vector were therefore reacted in the presence of 3 units of T4 ligase, at ambient temperature.

3) Transfection of Hepatocytes

The PGL3/PAI-1 plasmid is transfected into HepG2 cells. Transfection is carried out in plates having cells at 50% confluence, depositing Lipofectin® (1 mg/ml) and 1.5 μg of PGL3/PAI-1 plasmid in each of the wells. The Lipofectin® is activated for 30 min in an OPTI-MEM medium (GIBCO™) and then brought into contact for 15 min with the plasmids previously diluted with the medium. The cells are incubated for 6 hours at 37° C. in an atmosphere containing 5% CO₂ and 95% O₂. The transfection medium is withdrawn and replaced overnight by an enriched culture medium in order to stabilise the cells. Expression of the reporter genes is induced over 24 h in an MEM medium without serum. The cells are scratched and lysed in a lysis buffer (Dual-Luciferase® kit, Promega) and then held at −20° C.

The induction phase is 24 h for the HepG2 cells in the presence of TGFβ1 (1 ng/ml). During that induction phase, a PAI-1 expression inhibitor may be added 4 h before TGFβ1 and kept in contact with the cells until the end of the 24 h of the induction phase.

4) Determination of the Promoter Activity

The PAI-1 promoter activity is determined by quantification of the luciferase activity produced (Dual-Luciferase® kit, Promega). To each well there is added a solution of luciferin, the substrate of firefly luciferase. This results in an emission of light. The plate is incubated for 10 min in the dark because the luciferase activity is sensitive to light, and then a luminometer reading is started in order to quantify the photons emitted (Wallac, Perkin Elmer), the result obtained being the average cpm (counts per minute) over a period of 5 s.

5) Results

6,8-Diallyl-5,7-dihydroxy-2-(2-allyl-3-hydroxy-4-methoxyphenyl)-4H-1-benzopyran-4-one, arbitrarily referred to as compound A, and T686 of formula 3(E)-benzylidene-4(E)-(3,4,5-trimethoxybenzylidene)pyrrolidine-2,5-dione (PAI-1 expression inhibitor of reference, from Tanabe) were tested at a concentration of 10 μM on HepG2 cells in the basal state and after induction with TGFβ1. The PAI-1 promoter activity is measured in the control condition and in the presence of inhibitors, in the basal and induced states. The activities, expressed in cpm and as a percentage of the control observations, are shown in Table 1.

TABLE 1
Measurement of the PAI-1 promoter activity in the presence of
6,8-diallyl-5,7-dihydroxy-2-(2-allyl-3-hydroxy-4-methoxyphenyl)-
4H-1-benzo-pyran-4-one or T686 (24 h) in the basal condition or
the condition induced by TGFβ1 (1 ng/ml).

| | | | % |
|---|---|---|---|
| | | Activity in cpm | inhibition/control |
| Basal | Control | 1430 ± 59 | |
| | Compound A | 445 ± 121 | 69 |
| | (10 μM) | (**) | |
| | T686 (10 μM) | 473 ± 143 | 67 |
| | | (**) | |
| Induced (TGFβ) | Control | 3440 ± 242 | |
| | Compound A | 765 ± 138 | 78 |
| | (10 μM) | (°°) | |
| | T686 (10 μM) | 1641 ± 173 | 52 |
| | | (°°) | |

(**) p < 0.01 with respect to the control in the basal state,
(°°) p < 0.01 with respect to the control in the induced state.

ANOVA 1 factor, post Dunnett's test (n=6).

6,8-Diallyl-5,7-dihydroxy-2-(2-allyl-3-hydroxy-4-methoxyphenyl)-4H-1-benzopyran-4-one and T686 (10 μM) inhibit the expression of the PAI-1 gene in the basal condition and in the condition induced by TGFβ1. 6,8-Diallyl-5,7-dihydroxy-2-(2-allyl-3-hydroxy-4-methoxyphenyl)-4H-1-benzopyran-4-one (69%) is as potent as T686 (67%) in the basal condition and is moreover markedly more potent in the induced condition (78% inhibition as opposed to 52% for T686; p<0.01, ANOVA 1 factor, post Newman-Keuls test).

### EXAMPLE 2

### In Vivo Evaluation of the Antithrombotic Activity of Diosmetin Compounds

1) Animals

The studies were carried out on male CD rats weighing between 250 and 400 g (Charles River Laboratories). The experimental protocol carried out on groups of from 6 to 8 rats comprises a group for each compound under test to be compared with a control group in which the rats are treated with a solvent. These rats are used to study the effects of an antithrombotic in a model of arterial thrombosis induced by FeCl₃ on the abdominal aorta (Tanaka et al. Z-335, a new thromboxane A2 receptor antagonist, prevents arterial thrombosis induced by ferric chloride in rats, 2000, Eur. J. Pharmacol., 401, 413-418).

2) Model of Induced Arterial Thrombosis

The rats are treated with the compound under test, for example 6,8-diallyl-5,7-dihydroxy-2-(2-allyl-3-hydroxy-4-methoxyphenyl)-4H-1-benzopyran-4-one (30 mg/kg/day) by administration per os by gavage for 5 days before arterial thrombosis is induced in those rats. The rats are anaesthetised using pentobarbital 50 mg/kg by intraperitoneal administration and their abdominal aortas are exposed following laparotomy. An 8 mm-diameter pellet saturated with ferric chloride (FeCl₃ 50%) as causative agent is placed on the aorta for 10 minutes so as to damage the endothelium and induce the formation of a clot. In the 20 minutes following establishment of the model of arterial thrombosis, the clot formed is removed from the aorta and weighed.

3) Results

The model of arterial thrombosis induced by ferric chloride on the abdominal aorta in the rat makes it possible to verify the efficacy of diosmetin compounds according to the invention and in particular 6,8-diallyl-5,7-dihydroxy-2-(2-allyl-3-hydroxy-4-methoxyphenyl)-4H-1-benzopyran-4-one (compound A) as antithrombotic agents.

The activity of 6,8-diallyl-5,7-dihydroxy-2-(2-allyl-3-hydroxy-4-methoxyphenyl)-4H-1-benzopyran-4-one is evaluated as a function of the weight of the clot removed from the aorta, that activity being greater for a lesser weight of clot.

TABLE 2
Measurement of the activity of 6,8-diallyl-5,7-dihydroxy-2-
(2-allyl-3-hydroxy-4-methoxyphenyl)-4H-1-benzopyran-4-one
(compound A) as a function of the weight of the clot with
respect to the control (without gavage) (n = 6).

| | Weight of clot | |
|---|---|---|
| Control | 10.6 ± 0.7 | mg |
| Compound A | 7.6 ± 0.4 | mg |

6,8-Diallyl-5,7-dihydroxy-2-(2-allyl-3-hydroxy-4-methoxyphenyl)-4H-1-benzopyran-4-one makes possible a clear and significant reduction in the weight of the clot with respect to the control, the reduction in the clot being of the order of 30%.

1-6. (canceled)

7. A method of treating a living animal body, including a human, afflicted with thrombotic pathologies comprising the step of administering to the living animal body, including a human, an amount of a diosmetin compound of formula (I) 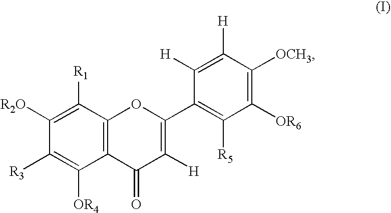
wherein:
R₁ represents a hydrogen atom or a propyl or allyl radical;
R₂ represents a hydrogen atom or a propyl, allyl, 2,3-dihydroxypropyl, (2,2-dimethyl-1,3-dioxol-4-yl)methyl or 3-acetyloxy-2-hydroxypropyl radical;
R₃ represents a hydrogen atom or a propyl or allyl radical;
R₄ represents a hydrogen atom or a methyl, propyl, allyl, 2,3-dihydroxypropyl or (2,2-dimethyl-1,3-dioxol-4-yl)methyl radical or a radical of formula —COR′₄ wherein R′₄ represents a linear or branched (C₁-C₅)alkyl radical or a phenyl radical;
R₅ represents a hydrogen atom or a propyl or allyl radical, and
R₆ represents a hydrogen atom or a methyl, propyl, allyl, 2,3-dihydroxypropyl or (2,2-dimethyl-1,3-dioxol-4-yl)methyl radical, a radical of formula —COR′₆ (wherein R′₆ represents a linear or branched (C₁-C₅)alkyl radical or a phenyl radical) or a radical of formula (I′) 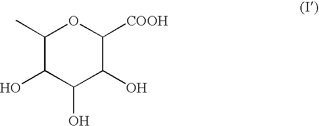
provided that:
at least one of the groups R₁, R₂, R₃, R₄, R₅ and R₆ is other than a hydrogen atom and,
if R₁, R₂ and R₃ each simultaneously represent a hydrogen atom, then R₄ also represents a hydrogen atom,
its diastereoisomers and enantiomers, and
addition salts thereof with a pharmaceutically acceptable acid or base,
which is effective for the prevention and/or treatment of thrombotic pathologies.

8. The method of claim 7 wherein the diosmetin compound is 6,8-diallyl-5,7-dihydroxy-2-(2-allyl-3-hydroxy-4-methoxyphenyl)-4H-1-benzopyran-4-one of formula (XVII):

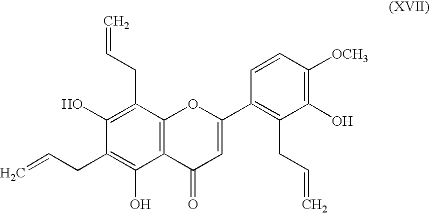

9. The method of claim 7, wherein the diosmetin compound is administered orally.

10. The method of claim 7, wherein the diosmetin compound is comprised with one or more pharmaceutically acceptable excipients in a pharmaceutical composition, which composition is administered for the prevention and/or treatment of thrombotic pathologies or pathologies with a risk of thrombosis.

11. A pharmaceutical composition comprising a diosmetin compound of claim 7 in combination with one or more pharmaceutically acceptable excipients, which composition is administered for the prevention and/or treatment of thrombotic pathologies or pathologies with a risk of thrombosis.


SEQUENCE LISTING (2 sequences)

SEQ ID NO: 1 - DNA, 27 bp, Artificial Sequence
  Primer
ttacgcgtgggtttggggctggacttg

SEQ ID NO: 2 - DNA, 28 bp, Artificial Sequence
  Primer
cgagatctcagaggtgccttgcgattgg